(19) United States
(12) Patent Application Publication
(10) Pub. No.: US 2025/0092120 A1
(43) Pub. Date: Mar. 20, 2025

(54) ANTICANCER PEPTIDE AND USE IN TREATING CANCER

(71) Applicant: A.T. STILL UNIVERSITY, Kirksville, MO (US)

(72) Inventor: James Lewis Cox, Kirksville, MO (US)

(21) Appl. No.: 18/886,444

(22) Filed: Sep. 16, 2024

Related U.S. Application Data
(60) Provisional application No. 63/582,710, filed on Sep. 14, 2023.

(51) Int. Cl. C07K 14/81; A61K 38/00; A61P 35/00; C07K 7/08; C12N 15/86

(52) U.S. Cl. CPC C07K 14/8139; A61P 35/00; C07K 7/08; C12N 15/86; A61K 38/00; C07K 2319/21

(57) ABSTRACT

A peptide, polypeptide, protein, DNA construct, vector, composition, or fusion protein for the treatment of a condition, such as cancer, and a method of treating the condition (cancer) using the peptide, polypeptide, protein, or composition, are disclosed. The polypeptide or composition includes a fusion protein comprising a modified cystatin peptide.

### CROSS REFERENCE TO RELATED APPLICATIONS

This application claims benefit of U.S. Provisional Application Ser. No. 63/582,710 filed Sep. 14, 2023, the entire contents of which is incorporated by reference herein.

### REFERENCE TO AN ELECTRONIC SEQUENCE LISTING

The contents of the electronic sequence listing (14715600039.xml; Size: 11,420 bytes; and Date of Creation: Sep. 16, 2024)) is herein incorporated by reference in its entirety.

### BACKGROUND

The current treatment of cancer is deficient in that durable remissions seldom occur for many patients. While immunotherapy has dramatically improved cancer treatment, immunotherapy works well for only a fraction of cancer patients. To improve beyond current treatments, new strategies and targets are required. Major challenges for cancer treatment to overcome include drug resistance, both intrinsic and acquired, and metastasis.

The cystatins belong to a superfamily of naturally occurring cysteine protease inhibitors[1]. Members of the cystatin superfamily are found in virtually all multicellular organisms and most tissues. The major target for inhibition by the cystatins are the cathepsins located in lysosomes of all eukaryotic cells. There are three basic types of cystatins: type I cystatins (stefins) type II cystatins, and type III cystatins (kininogens)[2]. Type I cystatins are mainly intracellular in location, type II are mainly secreted and are found in most body fluids, and type III are found at low levels in serum. The roles of the cystatins in cancer formation and progression are complex as cystatins can function as either tumor suppressors or tumor oncogenic factors depending on the type of cancer examined [3]. As cystatins are cysteine protease inhibitors, it might be imagined this activity confers the major anti-cancer effect of the cystatins, especially since virtually all cancers overexpress one or more cathepsins[4]. In reality, the cystatins possess several non-inhibitor activities which play a major role for anti-cancer effects of cystatins [5]. We have determined that overexpression of cystatin C in B16 F10 melanoma blocked metastasis in mice [6]. This mechanism involves a combination of inhibition of melanoma cell migration and induction of increased apoptosis of the melanoma cells in vivo [7]. We believe increased apoptosis of cancer cells upon cystatin overexpression is responsible for the greater part of metastasis inhibition. The mechanism of apoptosis induction in cancer cells by cystatin is still unclear. Although apoptosis increases several-fold upon cystatin overexpression, this overexpression does not result in massive cell death increases that would be required for dramatic tumor reduction. For this reason, we believe cystatin protein administration to tumor bearing animals cannot result in elimination of all cancer cells, but may result in reduced tumor burden compared to saline control animals. Through research in our laboratory, we found a short, 16mer cystatin peptide containing a highly conserved QVVAG sequence to contain most of the anti-metastatic activity of the cystatin protein sequence. However, a cystatin peptide or peptide composition with improved uptake efficiency, anti-cancer and anti-metastatic potency, and cell selection ability has yet to be identified. Therefore, a peptide or composition having these improved activities would be of great utility.

### SUMMARY

In one non-limiting aspect, a fusion protein is disclosed. The fusion protein includes a cystatin peptide, and a cell penetrating agent, for example a cell penetrating peptide, capable of penetrating a cell membrane. The fusion protein further includes a cleavable blocker peptide capable of inhibiting the cell penetrating peptide from penetrating the cell membrane when the cleavable blocker peptide is uncleaved, wherein the cleavable blocker peptide does not inhibit the cell penetrating peptide from penetrating the cell membrane when the cleavable blocker peptide is cleaved.

In a further aspect, a pharmaceutical composition that includes any of the fusion proteins disclosed herein is also provided.

In a further aspect, a polynucleotide comprising a sequence encoding any of the fusion proteins disclosed herein is provided.

In a further aspect, a virus that includes a viral genome that includes a sequence encoding any of the fusion proteins of this disclosure is also provided.

Another aspect is a method of treating a cancer, for example melanoma. The method includes administering to a subject a therapeutically effective amount of any of the pharmaceutical compositions disclosed herein.

In a further aspect, a method of treating a cancer-related condition is disclosed. The method includes administering to a subject a therapeutically effective amount of any of the pharmaceutical compositions disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a stained 10% acrylamide gel detecting the presence of a purified cystatin fusion peptide. In all cases, “cystatin fusion peptide” refers to N-terminal sequence cystatin peptide also containing a cell penetrating peptide, and a cleavable blocker peptide, and a C-terminal purification His-tag peptide, unless otherwise noted. The designation for a cystatin fusion peptide expressing yeast clone is 3XL1. Lane 1: Molecular weight markers. Lane 2: 4 μg purified cystatin fusion peptide. Lane 3: blank. Lane 4: 12 μg purified cystatin fusion peptide.

FIG. 2 is a graph illustrating the effect of the cystatin peptide on B16F10 melanoma cells via MTT assay. (Con)=control-no peptide. (CP)=cystatin fusion peptide.

FIGS. 3A-3E are live cell images of cystatin fusion peptide treated B16 melanoma cells. FIG. 3A shows 16 hours growth of control cells (10× objective). FIG. 3B shows treatment with fusion peptide for 1 hour followed by 15 hours in normal media resulted in some cell rounding with little apoptosis (10× objective). FIG. 3C shows 16 hours treatment with cystatin fusion peptide resulted in total cell death (fragmentation)(10× objective). FIG. 3D is continued incubation of control cells up to 40 hours total (40× objective), while FIG. 3E is 40-hour incubation following the 1 hour incubation with cystatin fusion peptide, showing a marked decrease in cell number compared to 40 hour control cells (40× objective).

FIG. 4 is a set of light micrographs illustrating the effect of the cystatin fusion peptide on B16F10 melanoma cells. (Con) control-no peptide. (CP) 40 μM cystatin fusion peptide.

FIG. 5 is a set of fluorescent micrographs illustrating B16F10 melanoma cells treated with either (top) no cystatin fusion peptide treated or (bottom) cystatin fusion peptide (40 uM). Cells were treated for 7 hours, then stained with propidium iodide (red), Mitotracker (green), and DAPI (nuclei, blue).

FIGS. 6A-6G show hematoxylin/eosin stained mouse tissue sections. FIG. 6A is mouse liver control, FIG. 6B is mouse 1 cystatin fusion peptide injected liver, and FIG. 6C is mouse 2 cystatin fusion peptide injected liver (10× objectives). FIG. 6D is mouse kidney control, FIG. 6E is mouse 1 cystatin fusion peptide injected kidney, and FIG. 6F is mouse 2 cystatin fusion peptide injected kidney (10× objectives). FIG. 6G shows cystatin fusion peptide treated mouse kidney section (40× objective).

### DETAILED DESCRIPTION

The present disclosure provides novel peptides, polypeptides, proteins, polynucleotides, DNA constructs, vectors, compositions, and methods of using the peptides, polypeptides, proteins, polynucleotides, DNA constructs, vectors, or compositions, including methods of treating a condition, which solve the issues raised in the disclosure. The peptides, polypeptides, proteins, polynucleotides, DNA constructs, vectors, and/or compositions may form or include all or part of a fusion peptide or protein as described. The fusion peptide or protein may be used in the disclosed methods.

As used in this specification and the claims, the singular forms “a,” “an,” and “the” include plural forms unless the context clearly dictates otherwise. For example, the term “a polypeptide fragment” should be interpreted to mean “one or more a polypeptide fragment” unless the context clearly dictates otherwise. As used herein, the term “plurality” means “two or more.”

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As described, peptides, polypeptides, proteins, polynucleotides, DNA constructs, vectors, compositions, and methods of using the peptides, polypeptides, proteins, polynucleotides, DNA constructs, vectors, or compositions, such as for the treatment of cancer and related condition or disease are disclosed. The polypeptides, proteins, polynucleotides, DNA constructs, vectors, and compositions relate to a fusion peptide or protein. The peptides, polypeptides, proteins, polynucleotides, DNA constructs, vectors, compositions, or fusion peptide/protein contain a peptide that is based upon at least a portion of a peptide sequence of a cystatin protein, such as cystatin C. The peptide, polypeptide, protein, polynucleotides, DNA construct, vector, or composition, or fusion protein may further include a cell-penetrating agent, such as a cell penetrating peptide. The polypeptide, protein, polynucleotide, DNA construct, vector, or composition, or fusion protein, may further include a blocking agent, such as a blocker peptide, for example a cleavable blocker peptide. The blocking agent, such as a blocker peptide or cleavable blocker peptide, prevents the activation of the cell-penetrating agent or cell-penetrating peptide until the blocking agent is removed (e.g., cleaved), such as by a protease, for example a tumor specific protease. One or more of a cystatin peptide, the cell penetrating peptide, and the blocker agent (or blocker peptide) may be linked to form a fusion protein. A polypeptide, protein, or composition containing the modified cystatin peptide or fusion protein can be delivered to a subject, such as a subject having a condition (for example a cancer), with a suitable carrier at a suitable dose.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like. As used herein, a “subject sample” or a “biological sample” from the subject refers to a sample taken from the subject, such as, but not limited to a tissue sample (e.g, fat, muscle, skin, neurological, tumor, etc.) or fluid sample (e.g., saliva, blood, serum, plasma, urine, stool, cerebrospinal fluid, etc.), and or cells or sub-cellular structures such as vesicles and exosomes. For example, the subject sample (e.g., a cell sample or cancer sample) may include a biopsy of a solid tumor from the subject.

In embodiments, the disclosed peptides, polypeptides, proteins, polynucleotides, DNA construct, vector, compositions, or fusion peptide or protein, may be used to treat a condition in a subject. In some embodiments, the condition may be a cancer. In some embodiments, the subject may be suffering from or diagnosed with cancer. By way of example, but not by way of limitation, a cancer treated by the peptides, polypeptides, proteins, compositions, pharmaceutical compositions, and fusion proteins of this disclosure may include metastatic cancers, breast cancer, colorectal cancer, lung cancer, prostate cancer, colon cancer, ovarian cancer, cervical cancer, gastrointestinal cancer, pancreatic cancer, glioma, glioblastoma, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, ovarian cancer, neuroblastoma, myeloma, various types of head and neck cancer, lymphoma cancer, leukemia, acute lymphoblastic leukemia, acute myeloid leukemia, Ewing sarcoma and peripheral neuroepithelioma, and other carcinomas, lymphomas, blastomas, sarcomas, and leukemias. For example, and in some embodiments, treating a subject with a pharmaceutical composition of the present disclosure may include treating metastatic disease, such as metastatic melanoma.

In some embodiments, the polypeptide, protein, polynucleotide, DNA construct, vector, or composition (e.g., pharmaceutical composition), or fusion protein, includes a protein or peptide sequence that inhibits cathepsins, for example a cystatin peptide. The amino acid sequence of the cystatin peptide may be based upon, or be associated with, a cystatin protein. For example, a cystatin peptide may include amino acid sequences from any of the type I, type II, or type III cystatins. For example, the cystatin peptide may be associated with the type II cystatin, cystatin C. The cystatin peptide may include amino acid sequences from one or more parts of a cystatin protein (e.g., an N-terminal amino acid sequence, a C-terminal amino acid sequence, or both). For example, the cystatin peptide may include a N-terminal amino acid sequence of human or murine cystatin C.

In embodiments, a composition may be or include a fusion protein containing the cystatin peptide as well as one or more other peptide, protein, or polypeptide sequence. The one or more peptide, protein, or polypeptide sequence that may be connected or fused to the cystatin peptide in the fusion protein may include but are not limited to a cell penetrating peptide, a blocker peptide, a purification peptide, (e.g., purification tag), and a marker peptide or marker peptide sequence (detection tag). For example, and in some embodiments, the cystatin peptide is linked to a cell penetrating peptide and a blocking peptide.

In embodiments, the polypeptide (fusion peptide/protein) includes SEQ ID NO: 2. SEQ ID NO: 2 is a fusion peptide or protein that includes a cystatin peptide sequence, a cell penetrating peptide sequence (penetratin peptide sequence), a cleavable blocker peptide sequence, and a purification tag sequence (his-tag peptide sequence). The fusion protein may include at least 60%, 70%, 75%, 80%, 85%, 90%, 95%, or 99% sequence identity to SEQ ID NO:2 or may include 60%, 70%, 75%, 80%, 85%, 90%, 95%, or 99% sequence identity to the cystatin peptide sequence (SEQ ID NO: 6), cell penetrating peptide sequence (SEQ ID NO: 5), cleavable blocker peptide sequence (SEQ ID NO: 3), and/or purification tag sequence (SEQ ID NO: 8). The fusion protein may be engineered with at least 1, 2, 3, 4, 5, 6, 7, 8, 9, or 10 amino acid substitutions to SEQ ID NO: 2 or to the cystatin peptide sequence (SEQ ID NO: 6), cell penetrating peptide sequence (SEQ ID NO: 5), cleavable blocker peptide sequence (SEQ ID NO: 3), and/or purification tag sequence (SEQ ID NO: 8).

In embodiments, a polypeptide, protein, DNA construct, vector, or composition, or fusion protein, may contain a purification tag. Purification peptide sequences or purification tags enable the peptide or fusion peptide to be produced in vivo and effectively purified using standard protein purification methods (e.g., affinity chromatography, FPLC, and HPLC). Purification peptide sequences fused in, or otherwise associated with the fusion protein include but are not limited to hexahistidine (His-tag) protein sequences, glutathione-S-transferase (GST) protein sequences, maltose binding protein (MBP), and streptavidin/avidin-based protein sequences. The fusion protein may further include more than one purification peptide sequences (purification tag). For example, the fusion protein may include a His-Tag protein sequence and a GST protein sequence.

The polypeptide, protein, DNA construct, vector, composition, or fusion peptide/protein may further include a marker peptide (detection tag). Marker peptide sequences enable the fusion protein to be identified in vivo (e.g., via immunofluorescence) or in situ. Examples of marker peptide or marker peptide sequences include but are not limited to peptide sequences for fluorescent proteins such as green fluorescent protein (GFP) and red florescent protein (RFP).

In some embodiments, the composition includes a cell penetrating agent fused to, or associated with, the cystatin peptide. The cell penetrating agent improves cellular uptake of the cystatin peptide. The composition may include any type of cell penetrating agent, including cell penetrating peptide (CPP). A CPP used may include, but not be limited to, a direct penetrating peptide (e.g., polycationic CPP), a peptide that mediates endocytosis (e.g., transactivating transcriptional activator (TAT)), translocation inducing peptide (e.g., MPG and Pep-1), and other peptides known to penetrate and cross cell membranes such as penetratin, including SynB1, SynB3, PTD-4, PTD-5, Transportan, MAP, SBP, FBP, Polyarginines, and Polylysines. The cell penetrating peptide may be any polypeptide having less than 50, 40, 30, or 20 amino acids that can penetrate cell membranes and deliver the fusion protein.

In some embodiments, the composition includes a blocking agent, such as a blocking peptide, fused to, or associated with, the fusion protein. The blocking agent inhibits the action of the cell penetrating agent or cell penetrating peptide (CPP), allowing the action of the cell penetrating agent, or CPP to occur in a cell-specific or tissue specific manner. For example, the composition may include a blocker peptide that is cleavable by specific proteases that are found intracellularly or extracellularly in the vicinity of a cancer cell or tumor. Once the blocker agent/peptide is cleaved by the protease, the CPP is activated (no longer constrained), and can enter a cell, preferably a targeted cell. Also, the cystatin peptide of the fusion protein is then able to enter the cell and/or exert a therapeutic action (e.g., cancer cell death).

The blocker peptide may be cleavable (e.g., a cleavable blocker peptide) by one of more proteases and/or have one or more of any protease cleavage sites including but not limited to protease sites for matrix metallopeptidase (MMPs) such as MMP1, MMP2, MMP3, MMP7, MMP8, MMP9, MMP10, MMP11, MMP13, MMP14, MMP15, MMP16, MMP17, MMP19, MMP20, MMP21, MMP23A, MMP 23B, MMP24, MMP25, MMP26, MMP27, and MMP28. For instance, the CPP may be bound to a blocker peptide that includes an MPP2 site. Once cleaved by MPP2, the CPP is no longer constrained, enabling the CPP to penetrate the cell, effectively and selectively delivering the peptide (e.g. cystatin peptide) or fusion peptide into the cell. The protease cleavage site may be incorporated into or adjacent to the blocker peptide. The use of alternative cleavage site peptides such as for cathepsins B or other proteolytic enzymes which are overexpressed on cancer cells would not substantially alter this invention.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., and a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. As to amino acid sequences, one of skill will recognize that individual substitutions to a peptide, polypeptide, or protein sequence which alters a single amino acid is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “fusion protein”, as used herein, refers to a polypeptide, peptide, or protein fused to another polypeptide, peptide, protein, or amino acid. For example, in this disclosure, the cystatin peptide linked to the cell penetrating peptide, is a fusion protein, and a cystatin peptide fusion protein contains the cell penetrating peptide and may also contain any one or more of a cleavable blocker peptide, a protease cleavage site peptide, a second blocker peptide, a purification tag, or any other peptide sequence.

The polypeptides or proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation, lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, enzymatic addition such as polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine) are also contemplated.

The polypeptides provided herein (e.g. cystatin polypeptides) may be full-length polypeptides or may be functional fragments of the full-length cystatin polypeptide. As used herein, a “fragment” “active fragment”, or “functional fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 120 contiguous amino acid residues of a reference polypeptide, respectively. In some embodiments, a fragment may comprise at least 5, 10, 15, 20, 25, 30, 40, 50, 60, 70, 80, 90, 100, or 110 contiguous amino acid residues of a reference polypeptide. Fragments may be preferentially selected from certain regions of a molecule. The term “at least a fragment” encompasses the full-length polypeptide. A fragment of a cystatin polypeptide may comprise or consist essentially of a contiguous portion of an amino acid sequence of the full-length cystatin polypeptide. A fragment of a cystatin polypeptide may include an N-terminal truncation, a C-terminal truncation, or both truncations relative to the full-length cystatin. Similarly, the fusion protein may include functional fragments of a full-length cystatin polypeptide, full-length cell penetrating peptide, and/or a full-length cleavable blocker peptide.

Regarding polypeptides or proteins, the phrases “percent identity,” “% identity,” and “% sequence identity” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 at least 150, or at least 171 contiguous residues. Such lengths are exemplary only, and it is understood that any fragment length supported by the sequences shown herein, in the tables, figures or Sequence Listing, may be used to describe a length over which percentage identity may be measured.

The amino acid sequences of the fusion protein and fusion protein variants as contemplated herein may include conservative amino acid substitutions relative to a reference amino acid sequence. For example, a variant or derivative fusion protein or fusion protein peptides may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The amino acid sequences of the fusion protein and fusion protein variants as contemplated herein may include modifications made apparent by a sequence alignment of the fusion protein or constituent peptides of the fusion disclosed herein. A person of ordinary skill in the art could easily align the fusion protein and/or constituent peptides of the fusion protein disclosed herein with polypeptides from, for example, other species to determine what additional variants (i.e., substitutions, insertions, deletions, etc.) could be made to the engineered fusion protein. For example, a person of ordinary skill in the art would appreciate that modifications in a reference fusion protein could be based on alternative amino acid residues that occur at the corresponding position in other homologous peptides from other species.

In some embodiments, the fusion protein includes a cell penetrating peptide that is linked via the N-terminus to a cleavable blocker peptide. In some embodiments, a cell penetrating peptide may be linked via the C-terminus to the cystatin peptide. In this manner, the organization of the fusion protein from N-terminus to C-terminus would include (1) cleavable blocker peptide, (2) cell penetrating peptide, and (3) cystatin peptide. Other peptide sequences (e.g., affinity tags, import sequences, second blocker peptide sequences) may be linked to the N-terminus of the cleavable blocker peptide and the C-terminus of the cystatin peptide, and may be inserted internally to the cleavable blocker peptide, the cell penetrating peptide, and the cystatin peptide.

In some embodiments, the fusion protein includes one or more synthetic amino acids, amino acid analogs and/or amino acid mimetics. For example, the fusion protein may include D-amino acids to confer peptide stability.

Isolated polynucleotides associated with or encoding the peptides and fusion proteins described herein are also provided. Those of skill in the art also understand the degeneracy of the genetic code and that a variety of polynucleotides can encode the same polypeptide. In some embodiments, the polynucleotides may be codon-optimized for expression in a particular cell such as, without limitation, a mammalian cell or a prokaryotic cell. While particular nucleotide sequences which are found in humans are disclosed herein, any nucleotide sequences may be used which encode a desired form of the substituted polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose as well as other means.

The isolated polynucleotides or polypeptides provided herein may be prepared by methods available to those of skill in the art. Isolated indicates that the polynucleotides or proteins are not in their naturally occurring state. Such preparations may be cell-free preparations. The polynucleotide or polypeptides may be extracted from the cells by breaking the cell membrane and optionally removing non-desired components. The polypeptides may be made as secreted polypeptides and further isolated using means known to those of skill in the art. Alternatively, desired proteins or nucleic acids can be purified using sequence-specific reagents, including but not limited to oligonucleotide probes, primers, and antibodies. Techniques for isolating cell-free preparations are well known in the art, and any that are convenient can be used. The term “substantially isolated or purified” refers to polypeptides or polynucleotides that are removed from their natural environment, and are at least 60% free, preferably at least 75% free, and more preferably at least 90% free, even more preferably at least 95% free from other components with which they are naturally associated.

In another aspect of the present disclosure, DNA constructs are provided. As used herein, the term “DNA construct” refers to recombinant DNA polynucleotides that may be single-stranded or double-stranded and may represent the sense or the antisense strand or both. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

The DNA constructs provided herein may be prepared by methods available to those of skill in the art. Notably each of the DNA constructs claimed are recombinant molecules and as such do not occur in nature. Generally, the nomenclature used herein and the laboratory procedures utilized in the present disclosure include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

Vectors, including any of the DNA constructs or polynucleotides described herein, are provided. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. Vectors may carry genetic elements, such as those that confer resistance to certain drugs or chemicals. For example, the DNA sequence encoding the fusion protein may be inserted into a plasmid for use as a mammalian expression vector. For instance, and in some embodiments, the DNA encoding the fusion protein may be inserted into a virus expression vector such as adenovirus (for example, pcDNA-3) for mammalian cell expression (e.g., the virus comprises a viral genome that includes the polynucleotide that encodes the fusion protein).

The DNA sequence of the vector or construct can also include a promoter which is recognized by the host organism and is operably linked to the fusion protein encoding nucleic acid. Promoters are untranslated sequences located upstream from the start codon of a structural gene (generally within about 100 to 1000 bp) that control the transcription and translation of nucleic acid under their control, including inducible and constitutive promoters. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known.

Pharmaceutical compositions including any of the fusion protein, polypeptides, polynucleotides, DNA constructs, or vectors described herein are provided. The pharmaceutical compositions may include a pharmaceutically acceptable carrier, excipient, or diluent (i.e., agents), which are nontoxic to the cell or mammal being exposed to the carrier, excipient, or diluent at the dosages and concentrations employed. Often a pharmaceutical agent is in an aqueous pH buffered solution. Examples of pharmaceutically acceptable agent include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ brand surfactant, polyethylene glycol (PEG), and PLURONICS™ surfactant. Examples of pharmaceutically acceptable carriers further include liposomes, micelles, artificial vesicles, and microspheres.

### Methods of Treatment

Methods of treating a condition and use of the disclosed peptides, polypeptides, proteins, polynucleotides, DNA constructs, vectors, and compositions are also provided. The methods may include administering any of the polypeptides, proteins, polynucleotides, DNA constructs, vectors, compositions or pharmaceutical compositions, or fusion proteins, as described herein to a subject in an amount effective to treat the condition. The condition may include any type of cancer, as described herein. For example and in embodiments, the method may include using the fusion protein or composition to inhibit tumour malignancy and/or tumour invasion.

As used herein, the terms “treat” and “prevent” are not intended to be absolute terms. In embodiments, treat and treatment can refer to a 10%, 20%, 30%, 40%, 50%, 60%, 70%, 80%, 90%, or 100% reduction in the severity of a tumour, established disease, condition, or symptom of the disease or condition. In embodiments, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. Treatment can refer to any delay in onset, amelioration of symptoms, improvement in patient skin appearance, etc. The effect of treatment can be compared to an individual or pool of individuals not receiving the treatment, or to the same patient prior to treatment or at a different time during treatment.

It will be appreciated that the specific dosage administered in any given case will be adjusted in accordance with the composition or compositions being administered, the disease to be treated or inhibited, the condition of the subject, and other relevant medical factors that may modify the activity of the compositions or the response of the subject, as is well known by those skilled in the art. For example, the specific dose for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compositions described herein and of a known agent, such as by means of an appropriate conventional pharmacological protocol.

The terms “effective amount,” “effective dose,” “therapeutically effective amount,” etc. refer to the amount of an agent that is sufficient to ameliorate a disorder, as described herein. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The peptides, polypeptides, proteins, polynucleotides, DNA constructs, vectors, compositions or pharmaceutical compositions, or fusion proteins, described herein may be administered one time or more than one time to the subject to effectively improve wound healing or other condition being treated. Suitable dosage ranges are of the order of several hundred micrograms effective ingredient with a range from about 0.01 to 10 mg/kg/day, preferably in the range from about 0.1 to 1 mg/kg/day. Precise amounts of effective ingredient required to be administered depend on the judgment of the practitioner and may be peculiar to each subject. It will be apparent to those of skill in the art that the therapeutically effective amount of the polypeptides, polynucleotides, and pharmaceutical compositions described herein will depend, inter alia, upon the administration schedule, the unit dose of antigen administered, whether the composition is administered in combination with other therapeutic agents, the status and health of the recipient, and the therapeutic activity of the particular composition.

Mode of administration may include intratumoral delivery, parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In one embodiment of the present disclosure, one would measure the clinical efficiency of the composition or fusion protein. For measurement of clinical therapeutic efficiency of the composition or fusion protein there are many imaging modalities that might be employed. One example would be MRI imaging of the tumour region in a patient to determine the relative growth or spread of the cancer before and after delivery of the peptide to a patient. An alternative would also be PET scan because of the high sensitivity of this imaging modality to cancer in the patient. Antibody methods of tumour detection are also possible, and although specialized techniques are required for their preparation, they are specific for certain cancers and hence within the realm of detection techniques that could be envisioned. One would consider the method of the present invention to be a success if tumour growth were halted, inhibited or reversed. Inhibition of growth of the metastatic tumour by at least 25% preferably 50%, may be considered a successful embodiment.

For melanoma examination a sentinel lymph node biopsy may be used to determine the peptide effect on status of the spread of the cancer. These biopsies are used for tumour staging in melanoma but could also give a critical window into progression during treatment with the described fusion protein or composition.

### Examples

The following examples are illustrative and should not be interpreted to limit the scope of the claimed subject matter.

### Identification of a Tumor Targeting Composition

An N-terminal cystatin C peptide is identified that potently induces cell death in melanoma cells when it comes in contact with melanoma cells. Cancer cell killing by this cystatin peptide is masked in intact cystatin because this action is greatly reduced with native cystatin protein. For a peptide to be efficiently taken up by cells it can be covalently linked to a cell penetrating peptide [8]. The cystatin peptide has been linked to penetratin, a well-known cell penetrating peptide [9]. For tumor targeting, a blocker peptide was also fused onto the cystatin peptide so that the penetrating peptide can be activated following cleavage and removal of the blocker peptide by tumor specific protease [10]. The final cystatin fusion peptide is expressed in yeast for affinity chromatography purification. The purified cystatin fusion peptide can be delivered to a cancer patient with a suitable carrier and at a suitable dose.

An anticancer peptide derived from cystatin C is described that is effective in killing cancer cells, such as melanoma cells by a mechanism that is probably apoptosis. This cystatin peptide has been further modified by adding a cell penetrating peptide sequence and cleavable blocker peptide that activates the penetrating peptide near tumor cells. This modified cystatin peptide (fusion peptide) can be delivered by any means to cancer cells, including intravenous injection, topical application to cancer cells, or injection directly into tumors.

Normal cells are generally zwitterionic, whereas cancer cells are often anionic in charge due to increased membrane associated phosphatidyl serine, proteoglycans, and/or sialylated glycoproteins in or on the cancer cell plasma membrane [11]. These negatively charged cancer cell membranes can selectively attract cationic peptides, particularly natural cationic antimicrobial peptides [12]. Many of these peptides can bind to cancer cell surfaces and actually penetrate cell membranes due to their amphipathic properties [13]. Different models have been suggested to provide a mechanism for pore formation and in vitro membrane preparations have been used to model cationic peptide actions [14]. When these peptides interact with microbial cell surfaces or cancer cell membranes and create pores in the surfaces or membranes, they are known as lytic peptides. There are however other types of cell death that can be induced in cancer cells [15]. Some peptides can enter into cancer cells and disrupt mitochondrial membranes resulting in apoptosis due to cytochrome c release [16-18]. Here, an N-terminal fragment of cystatin C is found to induce much higher levels of apoptosis than that occurring for the intact cystatin C protein. This latent cystatin peptide acts as an anticancer peptide much like lactoferricin, which is a proteolytic fragment of the milk protein, lactoferrin [19]. The mechanism of action is different between the two peptides in that lactoferricin actually lyses cancer cells by creating pores in the plasma membrane of the cells.

The mechanism of cell killing by the cystatin is probably apoptosis but necrosis cannot be ruled out as cell swelling is noted in melanoma cells after peptide treatment. Cystatin protein appears to contain a latent peptide that is cationic and belongs to a class of antimicrobial/anticancer peptides. For example, magainin, which is a small peptide derived from the skin of the African clawed toad, is able to lyse the membrane of human bladder cancer cells, but not fibroblasts [20]. There are a large number of these anticancer peptides which have been described, but this is the first report from a cystatin protein. While small anticancer peptides (20-50 amino acids) can be problematical with their proteolytic stability and short half-lives, a much larger peptide has been constructed to address these issues. Expression of an N-terminal peptide of cystatin C in B16 melanoma cells led to massive cell death of the melanoma cells after 2-3 days in a tissue culture dish with confluent cells. A DNA fragment was cloned, which was synthesized to correspond to the coding region of an N-terminal cystatin peptide.

Creation of pMET Alpha A-Cystatin Peptide Fusion Clone

pMETalpha, a plasmid DNA (Invitrogen), was digested with BamHI restriction enzyme to linearize the plasmid within the polylinker region. Linearized pMETalpha A (20 ng) was combined with a previously synthesized (IDT) DNA fragment (474 nucleotides) (20 ng) encoding murine cystatin C (60 amino acids, N-terminal region) linked to two other peptide sequences, a cell-penetrating peptide (penetratin) and a blocker peptide for penetratin which can be cleaved by tumor protease MMP2 (Jiang). Insertion of the cystatin fusion construct sequence into pMET alpha A plasmid allows one to take advantage of a yeast secretion signal for secretion of the cystatin peptide into the media of Pichia methanolica yeast cultures. In addition, a carboxy-terminal region in the yeast plasmid encodes a His-tag sequence that is used for peptide purification. This anticancer peptide includes five peptides linked together as synthesized by yeast. The secretion peptide is cleaved off during secretion of the peptide and hence is not found in the secreted, mature form of the peptide. The terminal histidine-tag (6×His-tag) sequence is part of a larger terminal peptide about 30 amino acids in length and is necessary for concentration and purification of the peptide from media after 3-4 days of yeast growth in shaker flasks with methanol feeding. For scale up of peptide production in yeast, it is of course possible to move to bio-fermenter production where essentially unlimited levels of this peptide could be isolated.

For cloning purposes, a g-block double-stranded DNA segment of 474 bps was synthesized by Integrated DNA Technologies (IDT). The nucleotide sequence of the disclosed fusion protein is the following:

(SEQ ID NO: 1)
ctcgagaaaagagaggctgaagctgaattcgaggagtccgctactg
gtgcttctaaaacggctaacccagtaacttcccaagaaccaacgg
gtgaacctgcatccacaggtgaagaatctgaaatcgaagaagctg
atggtagatctgatcatatggaaggtggtccgttaggtttagctg
gtcaattcagacaaatcaaaatctggttccctaatcgtcgtatga
agtggaagaaggcgtacgcgaccccaaaacaaggcccgcgaatgt
tgggagccccggaggaggcagatgccaatgaggaaggcgtgcggc
gagcgttggacttcgctgtgagcgagtacaacaagggcagcaacg
atgcgtaccacagccgcgccatacaggtgagagctcgtaagcagc
tggtggctggagtgaactatttttttgatgtgcatatggccgcca
gcttactagtaggtaagcct

The corresponding amino acid sequence of the fusion protein is the following:

(SEQ ID NO: 2)
GluAlaGluAlaGluPheGluGluSerAlaThrGlyAlaSerLysThr
AlaAsnProValThrSerGlnGluProThrGlyGluProAlaSerThr
GlyGluGluSerGluIleGluGluAlaAspGlyArgSerAspHisMet
GluGlyGlyProLeuGlyLeuAlaGlyGlnPheArgGlnIleLysIle
TrpPheProAsnArgArgMetLysTrpLysLysAlaTyrAlaThrPro
LysGlnGlyProArgMetLeuGlyAlaProGluGluAlaAspAlaAsn
GluGluGlyValArgArgAlaLeuAspPheAlaValSerGluTyrAsn
LysGlySerAsnAspAlaTyrHisSerArgAlaIleGlnValValArg
AlaArgLysGlnLeuValAlaGlyValAsnTyrPhePheAspValHis
MetAlaAlaSerLeuLeuValGlyLysProIleProAsnProLeuLeu
GlyLeuAspSerThrArgThrGlyHisHisHisHisHisHis.

The enzyme cleavage site sequence (e.g., MMP2), a cleavable site, which, when cleaved by cancer cell MMP2, releases blocker sequence from the cell penetrating peptide, is in bold only. The cell penetrating peptide sequence is underlined only. The cystatin C sequence (cystatin peptide) is italicized, bolded, and underlined. An N-terminal cleavable blocker sequence (cleavable blocker peptide) is in italics only; a C-terminal ‘degradation buffer’ sequence is also in italics only. A C-terminal purification tag (His tag) is italicized and underlined. The length of the peptide is 174 amino acids, about 20 kD in size.

As indicated above, the N-terminal blocker peptide includes a sequence of GluAlaGluAlaGluPheGluGluSerAlaThrGlyAlaSerLysThrAlaAsnProValThrSerGlnGluProThrGlyGluProAlaSerThrGlyGluGluSerGluIleGluGluAlaAspGlyArgSerAspHisMetGlu (SEQ ID NO: 3).

As indicated above, the enzyme cleavage site sequence is GlyGlyProLeuGlyLeuAlaGly (SEQ ID NO: 4).

As indicated above, the cell penetrating peptide of the fusion protein includes the peptide sequence GlnPheArgGlnIleLysIleTrpPheProAsnArgArgMetLysTrpLysLysAlaTyr (SEQ ID NO: 5), penetratin.

As indicated above, the cystatin peptide includes a peptide sequence AlaThrProLysGlnGlyProArgMetLeuGlyAlaProGluGluAlaAspAlaAsnGluGluGlyValArgArgAla LeuAspPheAlaValSerGluTyrAsnLysGlySerAsnAspAlaTyrHisSerArgAlalleGlnValValArgAlaArgLysGlnLeuValAlaGlyValAsnTyrPhePheAspVal (SEQ ID NO: 6).

As indicated above, the C-terminal sequence includes a peptide sequence of HisMetAlaAlaSerLeuLeuValGlyLysProIleProAsnProLeuLeuGlyLeuAspSerThrArgThrGly (SEQ ID NO: 7).

As indicated above, the carboxy terminal purification tag includes the His-Tag peptide sequence, HisHisHisHisHisHis (SEQ ID NO: 8). Other purification tags could be used such as GST, MBP, and streptavidin/avidin.

The C-terminal sequence may further include the peptide sequence HisMetAlaAlaSerLeuLeuValGlyLysProIleProAsnProLeuLeuGlyLeuAspSerThrArgThrGlyHisHisHisHisHisHis (SEQ ID NO: 9).

Transformation of pMAD11 Pichia methanolica Yeast

A colony of Pichia methanolica pMAD11 yeast (Invitrogen) was inoculated into 15 ml of yeast extract peptone adenine dextrose (YPAD) media and the culture was shaken at 30° C. until an OD600=0.1. Yeast cells were harvested by centrifugation and washed once in sterile TE (Tris 10 mM, EDTA 0.1 mM, pH7.4) buffer. Pelleted cells were resuspended in TE buffer plus 200 mM LiCl and incubated at 30° C. for one hour. Yeast cells were again pelleted and resuspended in 0.5 ml TE plus 200 mM LiCl to make competent cells. Competent cells (100 uL) were added to 3 ug pMETa-cystatin plasmid DNA and 50 ug denatured herring sperm DNA and allowed to incubate at 30° C. for 30 minutes. 100 μL of 70% polyethylene glycol was then added and mixed in and incubation continued for one hour at 30° C. Heat shock was then applied to the mixture at 42° C. for 5 minutes. The transformation mixture was then incubated at 4° C. overnight. The following day, the transformed yeast cells were plated on minimal dextrose (MD) plates and incubated for 5 days at 30° C. until colonies appeared. About 30 colonies resulted from the transformation and several were picked and grown for expression studies. One of the cystatin fusion peptide clones was chosen for all further work which we call clone 3XL1. Four 20 ml yeast cultures were grown for 24 hours in buffered dextrose-complex yeast medium (BMDY). Each of the four yeast cultures were then inoculated into 200 ml BMDY media (1 L flasks, fluted) for 24 hour shaking growth. Each 200 ml culture had yeast collected by centrifugation and resuspended in Buffered Methanol-complex Medium (BMMY) and 20 ml 5% methanol added once per day for 3 days shaking culture. Yeast were removed by centrifugation and spent media taken for further protein purification.

### His-Tag Purification of Cystatin Fusion Peptide

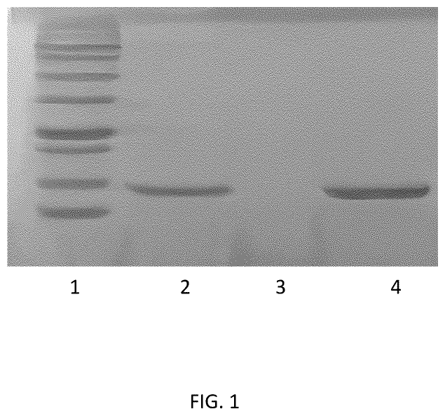

Yeast cell free, spent, methanol induced media was first adjusted to pH 7.2 with concentrated sodium hydroxide. The media was then incubated at 4° C. for two hours with 5 ml Talon (Takara) Cobalt beads for his-tag protein purification. All further work was done at 4° C. to reduce proteolysis. The beads were collected into plastic columns which were fitted with a filter to block bead flow into the effluent. The collected beads were washed with 10 bed volumes of sodium phosphate buffer (50 mM, pH 7.2, NaCl 300 mM). His-tag protein was eluted with 1 ml fractions of sodium phosphate buffer plus 0.15 M imidazole. Lowry protein assays were performed on 20 ul samples from each fraction. The peak fractions were pooled and dialyzed overnight against distilled water to remove phosphate, sodium chloride and imidazole. The dialyzed peptide solution was first frozen and then lyophilized and brought up to 2 mg per ml peptide with PBS. The peptide solution was then filter sterilized and frozen at −20° C. until before use. After protein assay, concentration of purified peptide solution was taken to 20 mg/ml final concentration and kept frozen at −80° C. prior to use. Some of the cystatin fusion peptide was run on PAGE gels to check for purity (FIG. 1).

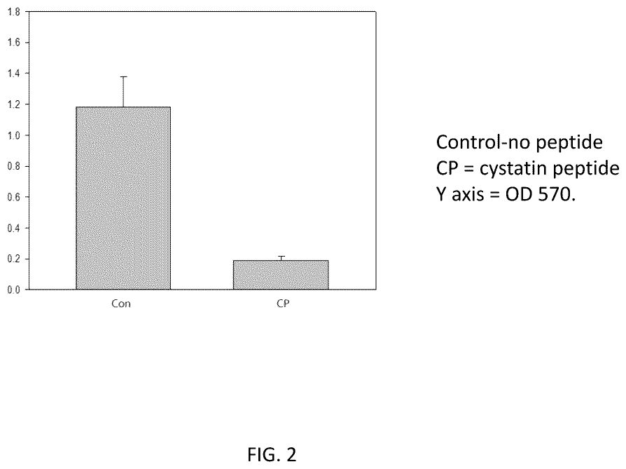

In order to test peptide effects on growth, B16F10 melanoma cells were plated in 96-well plates at 1×10⁴ cells per well. After 3 hours at 37° C., to 3XL1 peptide treated wells, 10 uM peptide was added in complete growth media. After 48-hour incubation, media was removed and replaced with growth media containing 10% MTT solution (5 mg/ml stock solution) for 4-hour incubation at 37° C. MTT-media solution was then aspirated and 100 ul isopropanol was added to each well and the plate was placed on a rocker for 15 minutes before reading at 570 nm in a spectrophotometer. FIG. 2 shows the results of 10 μM 3XL1 peptide treatment of B16F10 melanoma cells for 48 hours (Con=control-no peptide, CP=cystatin fusion peptide, 48 hours growth, Y axis is OD 570).

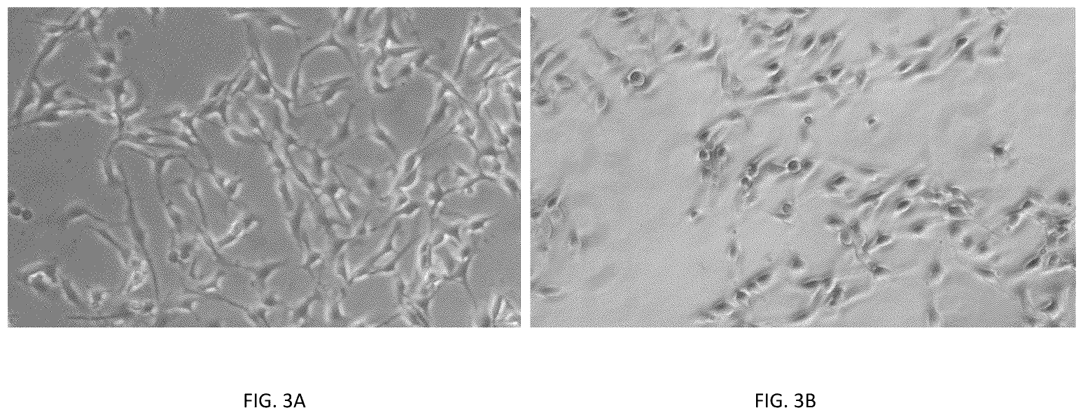

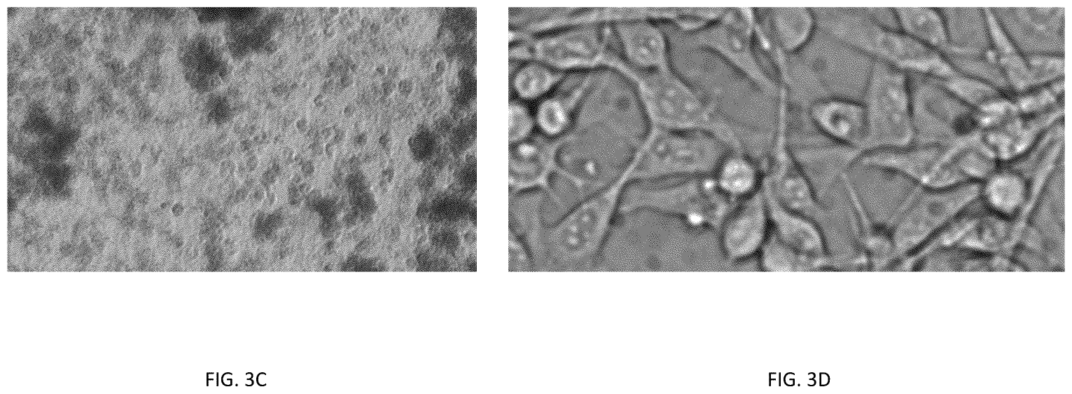

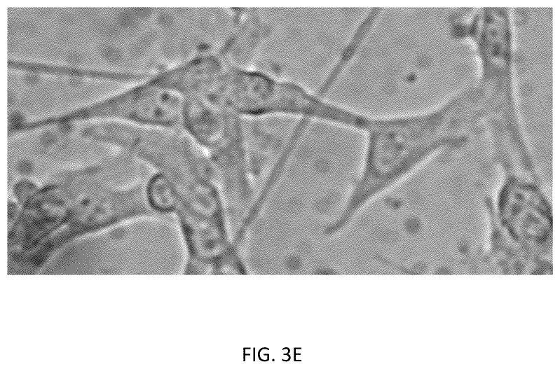

An experiment was conducted with live cell imaging of cystatin fusion peptide (3XL1) treated B16F10 melanoma cells in vitro. Twenty four well tissue culture plates were seeded with 4×10⁴ cells per well in RPMI 1640 media containing 10% FBS and antibiotics. The cells were allowed to attach and spread for 3 hours before addition of peptide. Cystatin fusion peptide was added at 20 μM concentration and either incubated for 16 hours or 1 hour, washed with media twice, and then standard media was added for 15 hours additional incubation. FIGS. 3A-3E show the results of cystatin fusion peptide treatment of the melanoma cells. Sixteen hours growth of control cells is shown in FIG. 3A (10× objective). Treatment with the cystatin fusion peptide for 1 hour followed by 15 hours in normal media resulted in some cell rounding but little apoptosis FIG. 3B (10× objective). Sixteen hours treatment with fusion peptide resulted in total cell death (fragmentation) with apparent apoptotic cell death FIG. 3C (10× objective). Continued incubation of control cells up to 40 hours total is shown in FIG. 3D (40× objective). Forty hour incubation following the 1 hour incubation with cystatin fusion peptide showed a marked decrease in cell number compared to 40 hour control cells FIG. 3E (40× objective).

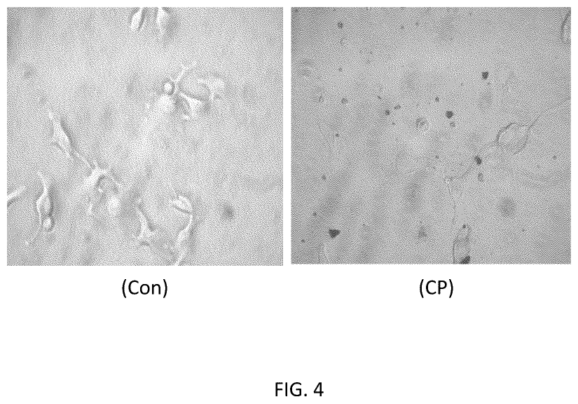

Further work involved testing the effect of cystatin fusion peptide (3XL1) on B16F10 melanoma cells in vitro. The melanoma cells, when grown in tissue culture media, appear flat with often cellular processes and extensions from a central cell body. When melanoma cells were treated with 40 uM cystatin fusion peptide for only 2 hours many of the cells had shrunken and condensed, which is often observed for cells undergoing apoptosis (FIG. 4). In a different experiment the cells were again treated with 40 uM cystatin fusion peptide for 7 hours, only this time cells were stained with propidium iodide to check for cell permeability, with Mitotracker™ to check for mitochondria staining, and with DAPI to check for nuclear condensation. Results showed some cell membrane permeability with propidium iodide staining, cell rounding and nuclear condensation suggesting apoptosis in a number of the cells (FIG. 5). The cystatin fusion peptide is not inhibiting cathepsins to produce apoptosis in the cancer cell. The reason is that cathepsins are known to trigger apoptosis in cell upon their release from lysosomes and cleavage of Bid protein which is then pro-apoptotic (Repnik,Stoka). Inhibition of cytosolic cathepsin by cystatin would block apoptosis by this same mechanism.

Delivery of cystatin protein to cancer cells does not have the same effect as delivery of the described cystatin fusion peptide. This is because the cell killing effect of the cystatin fusion peptide is latent in the cystatin protein unless released by proteolysis. This property is also seen for lactoferrin which has low cell killing activity. When the peptide lactoferricin is released by proteolysis, a much greater cell killing activity is seen with the shorter peptide [19].

In embodiments, the composition includes other homologues of cystatin C including the type I, II, and III cystatins and cystatin-like proteins, for example fetuins, cystatin-related epididymal spermatogenic (CRES) protein, and histidine-rich homologues. The composition may include any protein sequences with over 20% identity with cystatin C or other cystatins. The composition may include any peptide mimetic or chemical derivative that functionally relates to the disclosed cystatin fusion peptide sequence or cystatin peptide action. Short peptides can be partially or fully substituted with D-amino acids to confer peptide stability [21]. Substitution of one or more D-amino acids does not substantially change the cystatin peptide function except that biosynthesis of the peptide with D-amino acids would be very difficult.

A CPP used in this anticancer peptide is penetratin described by Derossi et al. [9]. CPPs interact with the cancer cell plasma membrane and translocate across the membrane along with any molecular cargo that is covalently attached to them. In this manner therapeutic cargoes, such as proteins, DNA, siRNAs, and anticancer drugs can be delivered into cancer cells. Obviously other CPPs could be substituted or added in some other combination or sequence that would not substantially alter the function of the composition. The CPP, penetratin, is required to allow for efficient uptake of the cystatin peptide into cells and hence traps the cystatin peptide in the cells the peptide comes in contact with after MMP2 cleavage by cancer cells [22].

In some embodiments of the disclosure, the composition is a nucleic acid molecule (polynucleotide) comprising the sequence, such as SEQ ID NO: 1, corresponding to the disclosed amino acids (SEQ ID NOs: 2-7). Also disclosed is delivery of such a nucleic acid molecule (polynucleotide) for the purposes of expression in cells. The cystatin peptide nucleic acid molecule could be delivered in the form of an expression plasmid to cancer cells or encapsulated in a virus or lipid-based chemistry to infect cancer cells, as is currently being done with certain oncolytic viruses.

One problem with short peptides as anticancer agents is the short half-life of the peptides due to proteolysis in vivo. A large anticancer peptide is disclosed that should have a longer half-life due to extra amino acids added to each end of the cystatin peptide, which serve as a buffer against endogenous peptidases. In other words, up to 30 amino acids can be removed from each end of the peptide without affecting the tumor cell killing property of the peptide.

### Therapeutic Application of the Peptide

In this example, the treatment of cancer is envisioned for this peptide against metastatic disease, the major cause of death from cancer (e.g., the peptide is used as a chemotherapeutic pharmaceutical composition). Therapeutic application of the described peptide is envisioned through intravenous delivery or infusion, such that there is delivery to systemic disease. Systemic delivery of chemotherapy is usually required for most cases of metastatic disease. Another route could be topical delivery to skin cancers or even direct injection of the cystatin fusion peptide in a suitable carrier into one or more tumors of the patient. Liposomal entrapment of the peptide for intravenous delivery is another method. Oral delivery would expose the peptide to damaging digestive enzymes, which limits the feasibility of that method unless the peptide could be protected.

Animal Experiment with Cystatin Fusion Peptide Treatment of Melanoma.

In this example, B16 melanoma tumor cells were first injected into C57B16 mice (Hilltop Laboratories). The B16F10 melanoma cells (ATCC) were collected from tissue culture dishes by trypsinization, and the trypsin was neutralized with an equal volume of growth media plus 10% FBS (fetal bovine serum). Collected cells were washed once in PBS (phosphate buffered saline) and then resuspended in PBS at 5×10⁵ cells per ml. Melanoma cells (1×10⁵ in 0.2 ml PBS) were injected with a syringe (30-gauge needle) into the lateral tail vein of all mice in the experiment. The injected mice were randomly divided into two groups: (1) PBS (0.2 ml) tail vein injected (10 mice) and (2) cystatin fusion peptide tail vein injected (1 mg cystatin fusion peptide in PBS/mouse/injection) (0.2 ml) (10 mice). Two injections in each of the groups of mice were on days 6 and 7 post-melanoma cell injections. On day 21 post-melanoma cell injections, all mice were euthanized by CO₂ inhalation and the lungs of the mice were excised for tumor enumeration. Table 1 shows the results of this experiment with pulmonary tumor counts in all the mice.

TABLE 1
Pulmonary tumor count in C57B16 mice 21 days
following B16 melanoma tail vein injection.

| Group | Lung tumor count |
|---|---|
| Control mice | 25, 42, 14, 2, 62, 34, 39, 16, 29, 21 |
| | Avg. 28.4 |
| Peptide treated mice | 0, 5, 3, 11, 14, 8, 5, 24, 4 |
| | Avg. 8.2 |

### Results

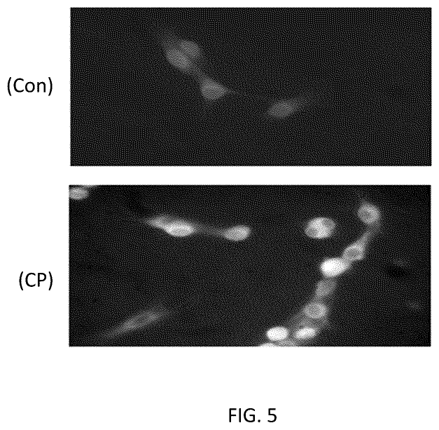

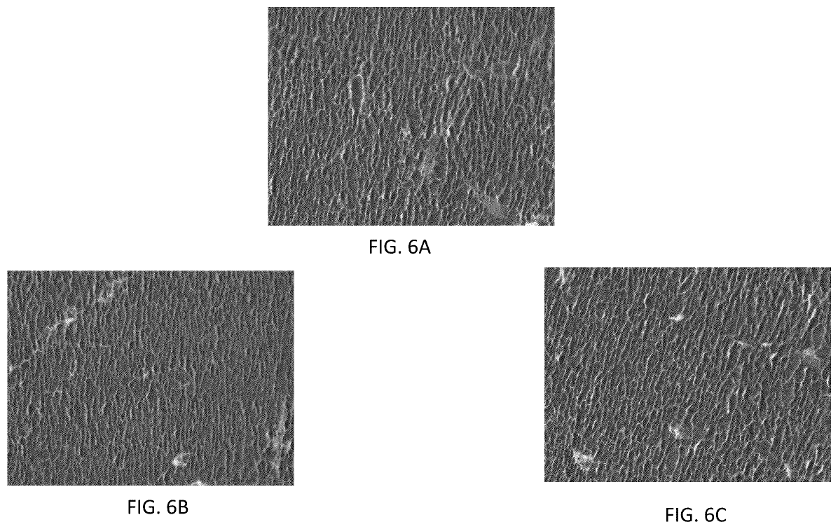

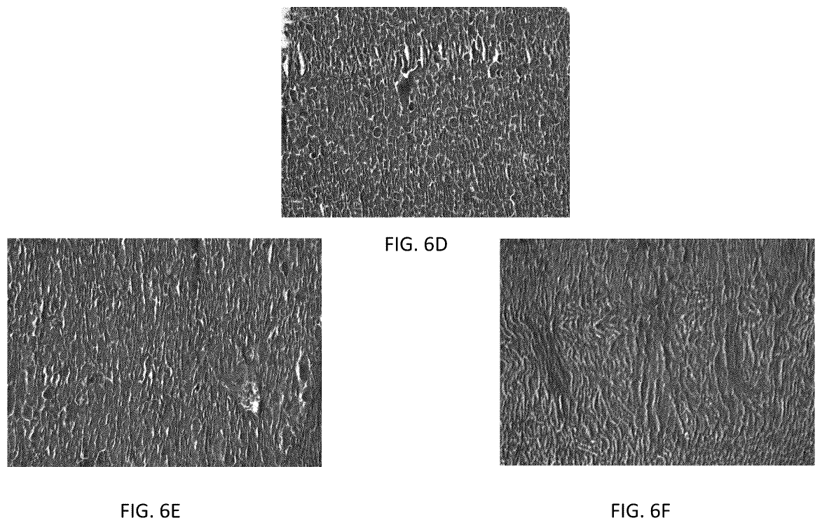

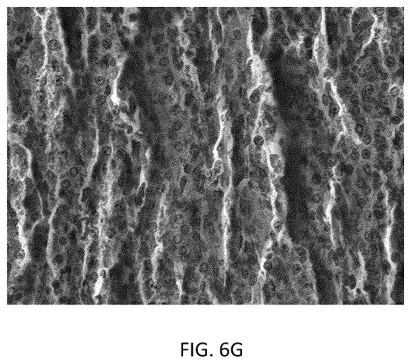

One mouse in the cystatin fusion peptide injected group died one day after first peptide injection. A 72% decrease in average melanoma tumor number resulted from 1 mg cystatin fusion peptide injections compared to saline control mice. One PBS injected and two random cystatin fusion peptide injected mice had their livers and kidneys sampled for histological analysis. FIGS. 6A-6F show light microscopic images (10× objective) of 4% paraformaldehyde fixed, paraffin embedded, 10 um sections. FIGS. 6A and 6D are control mouse liver and kidney respectively. FIGS. 6B and 6E are mouse 1, cystatin fusion peptide injected liver and kidney respectively. FIGS. 6C and 6F are mouse 2, cystatin fusion peptide injected liver and kidney respectively. No abnormalities were found in histological appearance of the cystatin fusion peptide injected mice tissue samples. FIG. 6G shows a kidney section (40× objective) of one of the peptide injected mice.

It will be readily apparent to one skilled in the art that varying substitutions and modifications may be made to the compositions and methods disclosed herein without departing from the scope and spirit of the compositions and methods. The compositions and methods illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the compositions and methods. Thus, it should be understood that although the present compositions and methods has been illustrated by specific embodiments and optional features, modification and/or variation of the concepts herein disclosed may be resorted to by those skilled in the art, and that such modifications and variations are considered to be within the scope of these compositions and methods.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

### REFERENCES

ADDIN ZOTERO_BIBL {“uncited”:[ ],“omitted”:[ ],“custom”:[ ]}CSL_BIBLIOGRAPHY 1. Turk V, Stoka V, Turk D. Cystatins: biochemical and structural properties, and medical relevance. Front Biosci. 2008; 13:5406-20.
2. Kordiš D, Turk V. Phylogenomic analysis of the cystatin superfamily in eukaryotes and prokaryotes. BMC Evol Biol. 2009; 9:266.
3. Lalmanach G, Kasabova-Arjomand M, Lecaille F, Saidi A. Cystatin M/E (Cystatin 6): A Janus-Faced Cysteine Protease Inhibitor with Both Tumor-Suppressing and Tumor-Promoting Functions. Cancers. 2021; 13:1877.
4. Mijanović O, Branković A, Panin A N, Savchuk S, Timashev P, Ulasov I, et al. Cathepsin B: A sellsword of cancer progression. Cancer Lett. 2019; 449:207-14.
5. Cox J L. Cystatins and cancer. Front Biosci Landmark Ed. 2009; 14:463-74.
6. Cox J L, Sexton P S, Green T J, Darmani N A. Inhibition of B16 melanoma metastasis by overexpression of the cysteine proteinase inhibitor cystatin C. Melanoma Res. 1999; 9:369-74.
7. Ervin H, Cox J L. Late stage inhibition of hematogenous melanoma metastasis by cystatin C over-expression. Cancer Cell Int. 2005; 5:14.
8. Liu S, Yang H, Wan L, Cheng J, Lu X. Penetratin-Mediated Delivery Enhances the Antitumor Activity of the Cationic Antimicrobial Peptide Magainin II. Cancer Biother Radiopharm. 2013; 28:289-97.
9. Derossi D, Calvet S, Trembleau A, Brunissen A, Chassaing G, Prochiantz A. Cell Internalization of the Third Helix of the Antennapedia Homeodomain Is Receptor-independent. J Biol Chem. 1996; 271:18188-93.
10. Jiang T, Olson E S, Nguyen Q T, Roy M, Jennings P A, Tsien R Y. Tumor imaging by means of proteolytic activation of cell-penetrating peptides. Proc Natl Acad Sci. 2004; 101:17867-72.
11. Manrique-Moreno M, Santa-González G A, Gallego V. Bioactive cationic peptides as potential agents for breast cancer treatment. Biosci Rep. 2021; 41:BSR20211218C.
12. Tornesello A L, Borrelli A, Buonaguro L, Buonaguro F M, Tornesello M L. Antimicrobial Peptides as Anticancer Agents: Functional Properties and Biological Activities. Molecules. 2020; 25:2850.
13. Takeshima K, Chikushi A, Lee K-K, Yonehara S, Matsuzaki K. Translocation of Analogues of the Antimicrobial Peptides Magainin and Buforin across Human Cell Membranes. J Biol Chem. 2003; 278:1310-5.
14. Haney E F, Mansour S C, Hancock R E W. Antimicrobial Peptides: An Introduction. In: Hansen P R, editor. Antimicrob Pept [Internet]. New York, NY: Springer New York; 2017 [cited 2023 Mar. 17]. p. 3-22. Available from: http://link.springer.com/10.1007/978-1-4939-6737-7_1
15. Ting C-H, Huang H-N, Huang T-C, Wu C-J, Chen J-Y. The mechanisms by which pardaxin, a natural cationic antimicrobial peptide, targets the endoplasmic reticulum and induces c-FOS. Biomaterials. 2014; 35:3627-40.
16. Hoskin D W, Ramamoorthy A. Studies on anticancer activities of antimicrobial peptides. Biochim Biophys Acta BBA—Biomembr. 2008; 1778:357-75.
17. Liu D, Angelova A, Liu J, Garamus V M, Angelov B, Zhang X, et al. Self-assembly of mitochondria-specific peptide amphiphiles amplifying lung cancer cell death through targeting the VDAC1-hexokinase-II complex. J Mater Chem B. 2019; 7:4706-16.
18. Zhang F, Angelova A, Garamus V M, Angelov B, Tu S, Kong L, et al. Mitochondrial Voltage-Dependent Anion Channel 1-Hexokinase-II Complex-Targeted Strategy for Melanoma Inhibition Using Designed Multiblock Peptide Amphiphiles. ACS Appl Mater Interfaces. 2021; 13:35281-93.
19. Yang N, Rekdal Ø, Stensen W, Svendsen J S. Enhanced antitumor activity and selectivity of lactoferrin-derived peptides: Antitumor activity of lactoferrin derived peptides. J Pept Res. 2002; 60:187-97.
20. Lehmann J, Retz M, Sidhu S S, Suttmann H, Sell M, Paulsen F, et al. Antitumor Activity of the Antimicrobial Peptide Magainin II against Bladder Cancer Cell Lines. Eur Urol. 2006; 50:141-7.
21. Werle M, Bernkop-Schnürch A. Strategies to improve plasma half life time of peptide and protein drugs. Amino Acids. 2006; 30:351-67.
22. Alves I D, Jiao C-Y, Aubry S, Aussedat B, Burlina F, Chassaing G, et al. Cell biology meets biophysics to unveil the different mechanisms of penetratin internalization in cells. Biochim Biophys Acta BBA—Biomembr. 2010; 1798:2231-9.

We claim:

1. A fusion protein comprising:
a cystatin peptide;
a cell penetrating peptide capable of penetrating a cell membrane; and
at least one cleavable blocker peptide capable of inhibiting the cell penetrating peptide from penetrating the cell membrane when the cleavable blocker peptide is uncleaved, wherein the cleavable blocker peptide does not inhibit the cell penetrating peptide from penetrating the cell membrane when the cleavable blocker peptide is cleaved.

2. The fusion protein of claim 1, comprising SEQ ID NO: 2.

3. The fusion protein of claim 1, wherein the cystatin peptide comprises at least an active fragment of cystatin C.

4. The fusion protein of claim 1, wherein the cystatin peptide comprises SEQ ID NO: 6.

5. The fusion protein of claim 1, wherein the cell penetrating peptide comprises a penetratin peptide.

6. The fusion protein of claim 1, wherein the cell penetrating peptide comprises SEQ ID NO: 5.

7. The fusion protein of claim 1, wherein the cleavable blocker peptide comprises a matrix metalloprotease 2 (MMP2) cleavage site.

8. The fusion protein of claim 1, wherein the cleavable blocker peptide comprises SEQ ID NO: 3 or SEQ ID NO: 4.

9. The fusion protein of claim 1, wherein the fusion protein further comprises a purification tag.

10. The fusion protein of claim 9, wherein the purification tag is a his-tag.

11. The fusion protein of claim 1, wherein the cystatin fusion protein comprises a detection tag.

12. A pharmaceutical composition comprising the fusion protein of claim 1.

13. The pharmaceutical composition of claim 12, further including a pharmaceutically acceptable carrier.

14. The pharmaceutical composition of claim 13, wherein the pharmaceutically acceptable carrier comprises a liposome, a micelle, or a microsphere.

15. The pharmaceutical composition of claim 12, wherein the fusion protein is encapsulated in a virus.

16. A polynucleotide comprising a sequence encoding the fusion protein of claim 1.

17. A virus comprising a viral genome, wherein the viral genome comprises the polynucleotide of claim 16.

18. A method of treating melanoma comprising administering to a subject a therapeutically effective amount of the pharmaceutical composition of claim 12.

19. A method of treating melanoma comprising administering to a subject a therapeutically effective amount of the virus of claim 17.

20. A method of treating a cancer-related condition comprising administering to a subject a therapeutically effective amount of the pharmaceutical composition of claim 12.


SEQUENCE LISTING (9 sequences)

SEQ ID NO: 1 - DNA, 471 bp, synthetic construct
  source 1..471: mol_type = other DNA
ctcgagaaaagagaggctgaagctgaattcgaggagtccgctactggtgcttctaaaacggctaacccagtaacttcccaagaaccaacgggtgaacctgcatccacaggtgaagaatctgaaatcgaagaagctgatggtagatctgatcatatggaaggtggtccgttaggtttagctggtcaattcagacaaatcaaaatctggttccctaatcgtcgtatgaagtggaagaaggcgtacgcgaccccaaaacaaggcccgcgaatgttgggagccccggaggaggcagatgccaatgaggaaggcgtgcggcgagcgttggacttcgctgtgagcgagtacaacaagggcagcaacgatgcgtaccacagccgcgccatacaggtgagagctcgtaagcagctggtggctggagtgaactatttttttgatgtgcatatggccgccagcttactagtaggtaagcct

SEQ ID NO: 2 - protein, 174 aa, synthetic construct
  REGION 50..57: note = enzyme cleavage site
  REGION 58..75: note = cell penetrating sequence
  REGION 76..143: note = cystatin C sequence
  REGION 144..168: note = N-terminal cleavable blocker sequence
  REGION 169..174: note = His tag
EAEAEFEESATGASKTANPVTSQEPTGEPASTGEESEIEEADGRSDHMEGGPLGLAGQFRQIKIWFPNRRMKWKKAYATPKQGPRMLGAPEEADANEEGVRRALDFAVSEYNKGSNDAYHSRAIQVVRARKQLVAGVNYFFDVHMAASLLVGKPIPNPLLGLDSTRTGHHHHHH

SEQ ID NO: 3 - protein, 49 aa, synthetic construct
EAEAEFEESATGASKTANPVTSQEPTGEPASTGEESEIEEADGRSDHME

SEQ ID NO: 4 - protein, 8 aa, synthetic construct
GGPLGLAG

SEQ ID NO: 5 - protein, 20 aa, synthetic construct
QFRQIKIWFPNRRMKWKKAY

SEQ ID NO: 6 - protein, 66 aa, synthetic construct
ATPKQGPRMLGAPEEADANEEGVRRALDFAVSEYNKGSNDAYHSRAIQVVRARKQLVAGVNYFFDV

SEQ ID NO: 7 - protein, 25 aa, synthetic construct
HMAASLLVGKPIPNPLLGLDSTRTG

SEQ ID NO: 8 - protein, 6 aa, synthetic construct
HHHHHH

SEQ ID NO: 9 - protein, 31 aa, synthetic construct
HMAASLLVGKPIPNPLLGLDSTRTGHHHHHH